(19) United States
(12) Patent Application Publication
(10) Pub. No.: US 2004/0030295 A1
(43) Pub. Date: Feb. 12, 2004

(54) Safety syringe

(76) Inventor: Adrian Corbett, San Diego, CA (US)

Correspondence Address: Karl M. Steins, Steins & Associates, Suite 120, 2333 Camino del Rio South, San Diego, CA, 92108, US

(21) Appl. No.: 10/213,457

(22) Filed: Aug. 6, 2002

(51) Int. Cl. A61M 5/32

(52) U.S. Cl. 604/198; 128/919

(57) ABSTRACT

A Safety Syringe is disclosed. Also disclosed is a syringe that includes an annular reservoir that permits an extendable needle guard to be stowed therein. The diameter of the syringe body is greater than the diameter of the needle guard. The guard may be actuated to cover the syringe needle by depressing a lever or button located adjacent to the plunger end of the syringe. The release lever or button further is provided with a release guard to prevent inadvertent triggering of the needle guard. The syringe further includes a sealing ring at the tip end of the plunger that serves to seal the annular reservoir for retaining fluid therein. Furthermore, an alternate design of the syringe is provided that has an asymmetrical tip on both the sealing ring and the annular reservoir in order to inhibit the formation of bubbles at the tip end of the syringe.

### BACKGROUND OF THE INVENTION

1. Field of the Invention

This invention relates generally to medical products and, more specifically, to a Safety Syringe

2. Description of Related Art

Accidental puncturing one's self or others with a drug syringe needle, known as “needlesticks” is now recognized as a serious health problem by the health care community. In response, medical device manufacturers have developed a variety of different “safety syringe” designs that all include some sort of protective cover or mechanism for their needles. Examples of the most pertinent prior safety syringes include: Grabis, et al., U.S. Pat. No. 6,322,540, Sanpietro, U.S. Pat. No. 5,562,626 and Castagna, U.S. Pat. No. 5,411,487.

The Grabis, Sanpietro, and Castagna patents all disclose a “safe needle device for syringe” which include an outer tubular protective sleeve for sliding over the outside of the syringe and needle upon completion of use of the syringe. Although the outer sleeve design of these patents may seem to be functional, it in fact is an over-sized package that is difficult to grasp and use. Furthermore, most or all of the designs require complex manufacturing and assembly that result in a cost-prohibitive final product. Still further, each of these prior designs requires two hands for operation, making the devices more cumbersome (and possibly unsafe) than the conventional (non-safe needle syringes). Also, these prior styles present difficulties with reading the graduations since you must look through the sliding sleeve to see the marks (i.e. the graduations). Finally, the exterior guards of the prior styles may interfere with the drawing and dispensing fluids and may result in inadvertent releases. What is needed is a low-cost yet reliable device for aiding in the prevention of needlesticks.

### SUMMARY OF THE INVENTION

In light of the aforementioned problems associated with the prior devices and systems, it is an object of the present invention to provide a Safety Syringe. The syringe should include an annular reservoir that permits an extendable needle guard to be stowed therein. The diameter of the syringe body should be greater than the diameter of the needle guard. The guard should be actuated to cover the syringe needle by depressing a lever or button located adjacent to the plunger end of the syringe. The release lever or button should further be provided with a release guard to prevent inadvertent triggering of the needle guard. The syringe should further include a sealing ring at the tip end of the plunger that serves to seal the annular reservoir for retaining fluid therein. It is a further object that an alternate design be provided that has an asymmetrical tip on both the sealing ring and the annular reservoir in order to inhibit the formation of bubbles at the tip end of the syringe.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings, of which:.

FIGS. 1A-1D are side views of one embodiment of the safety syringe of the present invention;

FIG. 2 is a cutaway side view of the body of the syringe of FIGS. 1A-1D;

FIG. 3 is a cutaway side view of the plunger of the syringe of FIGS. 1A-1D;

FIG. 4 is a cutaway side view of the guard assembly of the syringe of FIGS. 1A-1D;

FIG. 5 is an end view of an alternate embodiment of the syringe of the present invention further including a release guard;

FIGS. 6A-6C are cutaway side views of the operation of the syringe of FIG. 5; and

FIG. 7 depicts cutaway side views of an embodiment of the asymmetrical tip versions of the body and plunger of the present invention.

### DETAILED DESCRIPTION OF THE PREFERRED EMBODIMENTS

The following description is provided to enable any person skilled in the art to make and use the invention and sets forth the best modes contemplated by the inventor of carrying out his invention. Various modifications, however, will remain readily apparent to those skilled in the art, since the generic principles of the present invention have been defined herein specifically to provide a Safety Syringe.

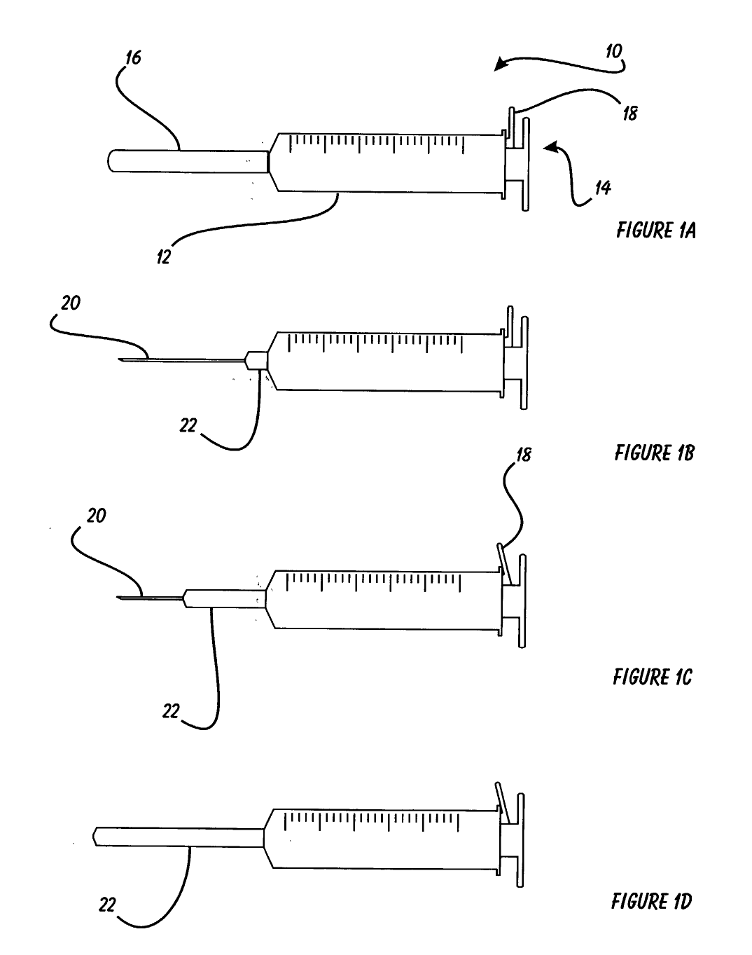

The present invention can best be understood by initial consideration of FIGS. 1A-1D. FIGS. 1A-1D are side views of one embodiment of the safety syringe 10 of the present invention. The syringe 10 comprises a body 12, a plunger 14, and a guard release member 18 extending radially from the plunger end of the syringe 10 (i.e. opposite from the needle end). The cap 16 is shown installed as it might be when the syringe is first taken from its protective packaging for use.

Once the cap 16 is removed, the needle 20 and tip end of the guard 22 are exposed. It should be noted immediately that the guard 22 of the present invention does not surround the circumference of the body 12, but rather is retained within it; this is clearly in contrast with the prior safety syringe designs.

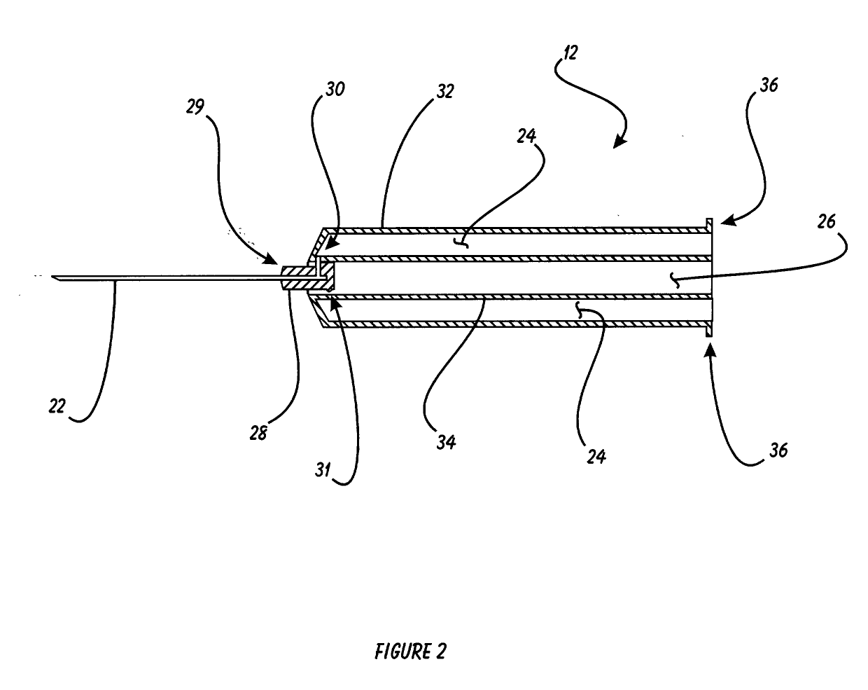

FIG. 1C depicts the syringe 10 after its use is complete (i.e. its contents have been dispensed). Immediately upon removal of the needle 20 from the skin (or perhaps even as the needle 20 is being withdrawn from the skin), the guard release member 18 is manually depressed by the user. Depressing the release member 18 an adequate amount releases the guard 22 and causes it to extend over the length of the needle 20 (shown partially extended in FIG. 1C). As depicted in FIG. 1D, the guard 22 will come to rest after fully extending to cover the entire length of the needle 20, and it will automatically be locked in this position. The entire elapsed time from depression of the guard member 18 until the guard 22 is locked in place as shown in FIG. 1D is fractions of a second; it has been depicted in two figures here simply to aid in the discussion of the operation of the device 10. Now turning to FIG. 2, we can begin to examine the individual components of the present invention.

FIG. 2 is a cutaway side view of the body 12 of the syringe of FIGS. 1A-1D. A significant feature of the body 12 is the existence of a doughnut-shaped (as viewed from the rear) annular reservoir 24 between an outer wall 32 and an inner wall 34. This annular reservoir 24 is an approach unlike anything previously found, and it provides substantial utility. Specifically, because the center (radially) region of the body 12 is not a part of the reservoir 24, it is not a part of the fluid chamber of the syringe. As a result, this center region can be used as the guard chamber 26, or the area within which many of the components of the needle guard (not shown) are held. The specific components of the needle guard assembly will be discussed in connection with subsequent drawing figures.

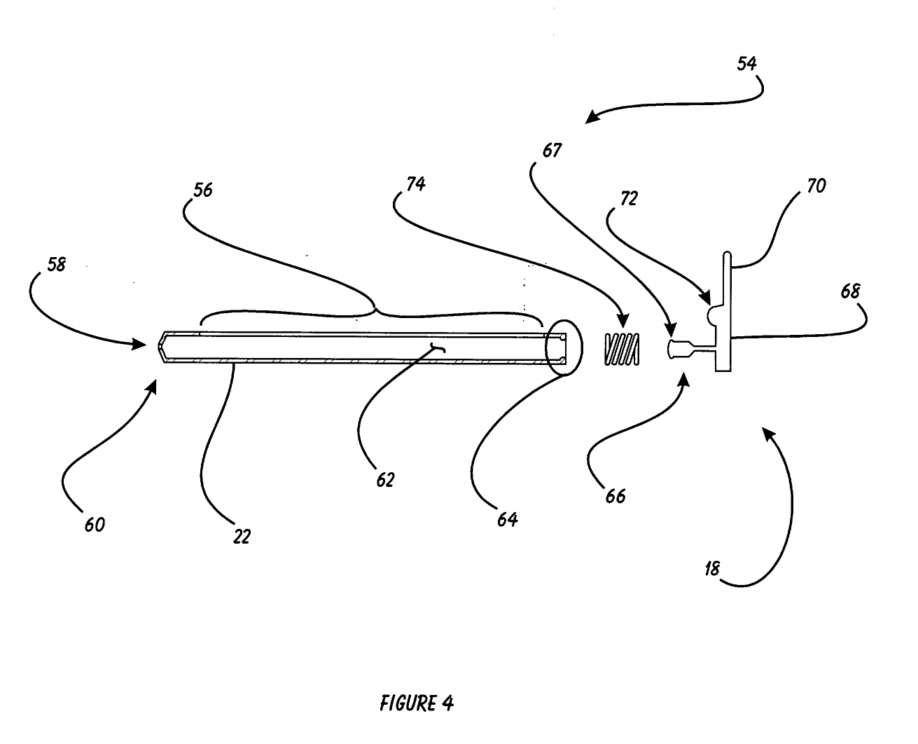

The body 12 further includes a needle base 28 from which the needle 22 extends, and which is further defined by a ridge 31 formed therein. The ridge 31 is provided to cooperate with a ridge formed on the guard (see FIG. 4) to lock the guard (see FIG. 4) once the guard has extended. The needle base 28 is further defined by a central bore 29 (which is in fluid communication with the central bore of the needle 22) that is placed in fluid communication with the annular reservoir by a radial port 30 extending substantially radially from the central bore and into the reservoir 24. In a non-depicted design, there is a pair of radial ports 30 on opposite sides of the annular reservoir, which will provide additional support to the base of the needle.

It should be seen that fluid forced down the reservoir 24 towards the needle end of the body 12 will be forced through the radial port 30, through the central bore 29, and out through the needle 22. Clearly, this design is different and superior to those that have come before it.

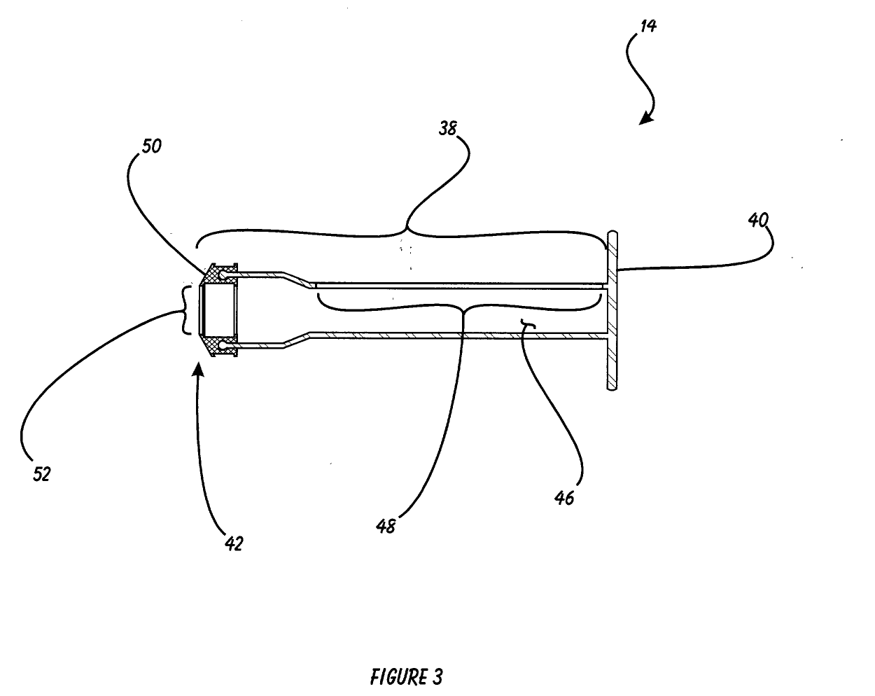

Also shown in this diagram is a ridge 36 running around the open end of the body to assist the user in grasping the syringe, similar to the conventional (non-safety) syringe. If we now turn to FIG. 3, we can continue to examine the individual elements of this advanced safety syringe.

FIG. 3 is a cutaway side view of the plunger 14 of the syringe of FIGS. 1A-1D. The plunger 14 comprises a plunder stem 38 and a sealing ring 50. The plunger stem 38 is defined a first end 40 and a second end 42. The pliable, rubberized sealing ring 50 extends from the second end 42 of the plunger stem 38. A flat, generally circular button is formed at the first end 40 of the plunger stem 38. The sealing ring 50 has an aperture 52 at its center (axially) such that it can provide a positive seal against both the outer wall and inner wall of the body (see FIG. 2). The sealing ring 50 takes the functional place of the conventional rubberized plug or tip found on a typical non-safety syringe, i.e. it is used to force fluid out through the needle (or, alternatively, to draw it in).

The plunger stem 38 forms a stem void 46 at its center for accommodating the components of the guard assembly as is discussed more fully below. Furthermore, the stem 38 includes a release member slot 48 formed along a substantial portion of the length of the plunger stem 38 to permit the guard release member (see FIGS. 1, 4 and 6) to protrude outwardly from the stem void 46 such that it can be reached by the user. Now turning to FIG. 4, we can examine the details of the novel guard of the present invention.

FIG. 4 is a cutaway side view of the guard assembly 54 of the syringe of FIGS. 1A-1D. The guard 22 is sized to extend over the entire length of the needle when it is in its fully extended position (see FIG. 1). The guard 22 is defined by a guard void 62 formed at the center of the guard 22 that is sized to accept the needle base (see FIG. 1) therewithin. The guard void 62 terminates in a substantially closed tip 60 at one end, and in a substantially open other end. It must be pointed out here that in a non-depicted version, the needle would be replaceable (e.g. to change needle size); in these versions, the tip end of the guard 22 would be open (rather than substantially closed) in order to permit the user to install and remove needles.

The substantially open end is further defined by a ridge 64 formed to partially or fully encircle the opening. Details of the purpose of this ridge 64 feature will be discussed further below.

The tip 60 is further defined by a needle aperture 58 through which the needle can extend (when the guard 22 is in the retracted position). Furthermore, the guard 22 is defined by a needle port slot 56 extending substantially along its length. The slot 56 is sized to permit the radial port portion of the needle base (see FIG. 2) to extend therethrough to permit the guard 22 to slide in and out of the syringe without interfering with the needle base (see FIG. 2).

The assembly 54 also includes a biasing member 74, such as the coil spring shown here (in a compressed condition). The biasing member 74 functions to force the guard 22 to rapidly extend over the needle once the guard release member 18 is depressed. The coil spring biasing member 74 includes an open center area as with conventional coil springs (although not depicted here).

The guard release member 18 is defined by a lever 68 that terminates in a button 70 near one end, and a guard-engaging portion 66 extending from an area near its other end. The circumference of the guard-engaging portion 66 is sized and shaped such that it can pass without restriction through the center portion of the coil spring biasing member 74, and into the void 62 of the guard 22. The guard-engaging portion 66 is further defined by a ridge 67 formed substantially around its entire circumference. The ridge 67 is provided to securely engage the ridge 64 formed at the open end of the guard 22.

It should be intuitively apparent that the syringe is assembled by depressing the guard 22 to compress.,the biasing member 74 (as it is shown here) until the guard-engaging portion 66 of the guard release member 18 passes through the entire length of the compressed biasing member 74 and into the open end of the guard 22. The ridge 67 consequently engages the ridge 64, and the guard 22 is restrained in this position.

In order to release the guard 22, the user need simply to press down on the button 70 formed at the distal end of the lever 68. This will cause the lever 68 to rock on a 20 fulcrum 72 (or similar protrusion) extending from the lever 68; when sufficient pressure is applied to the button 70, the ridge 67 will be pulled past the ridge 64, and the guard 22 will be released from the guard-engaging portion 66. Once the guard 22 is released from the guard-engaging portion 66, the biasing member 74 will push the guard 22 away from the guard release member 18 (which is retained within the body of the syringe) until the guard 22 is fully extended (referred to as the extended position).

It should be noted that in another “triggerless” embodiment, the guard 22 is released by simply pressing the plunger down to a fully-depressed condition, thereby causing a modified guard-engaging portion to release the guard 22.

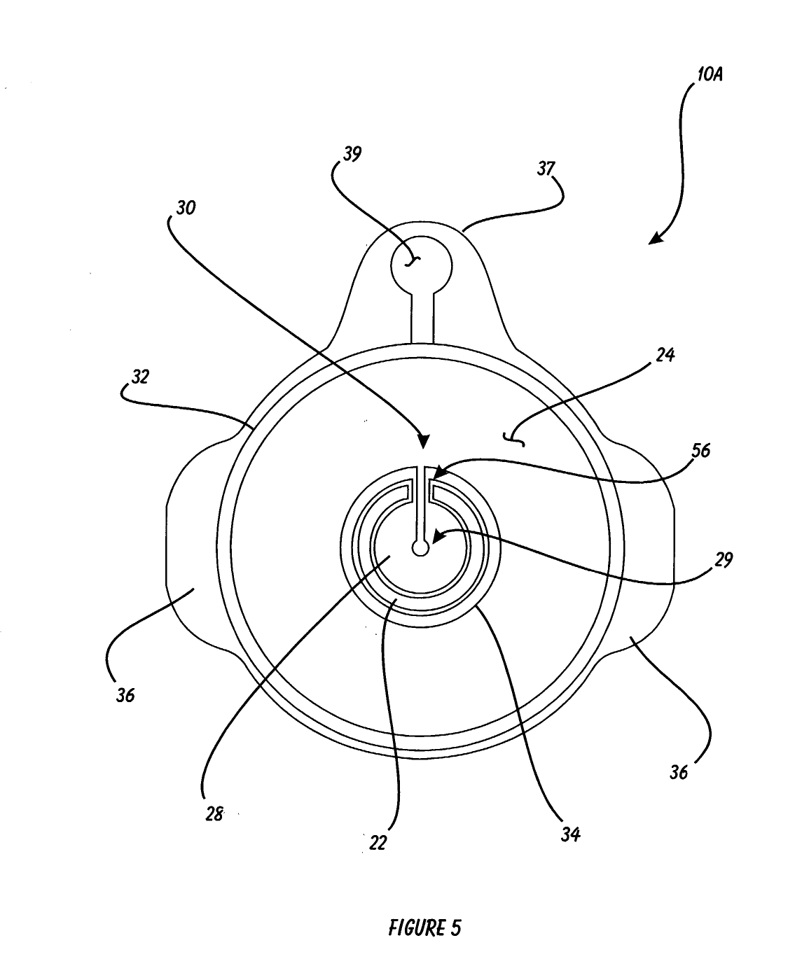

The ridge 64 provides additional functionality, namely, to lock the guard 22 in place once it is fully extended. A corresponding ridge or groove is provided on the outer periphery of the needle base (see FIG. 2); the ridge or groove provided in the needle base will permit the ridge 64 to pass by it as the guard 22 is extending, but it will prevent the ridge 64 from passing in the opposite direction. In this manner, the guard will lock in place, fully covering the entire length of the needle. If we now turn to FIG. 5, we can examine the syringe from another perspective in order to gain additional insight into its novel design.

FIG. 5 is an end view of an alternate embodiment of the syringe 10A of the present invention further including a release guard 37. The release guard 37 is an extension from the ridge 36 extending from the body of the syringe 10. The release guard 37 is defined by an aperture 39 formed therein to accept the lever and button areas of the guard release member (see FIG. 4). Because the release guard 37 extends radially beyond the outer edge of the guard release member, it will serve to substantially prevent the inadvertent triggering of the guard release. The aperture 39 and button (of the guard release member) may be cooperatively designed such that there is a slight interference fit between the two to further inhibit the unintentional triggering of the guard.

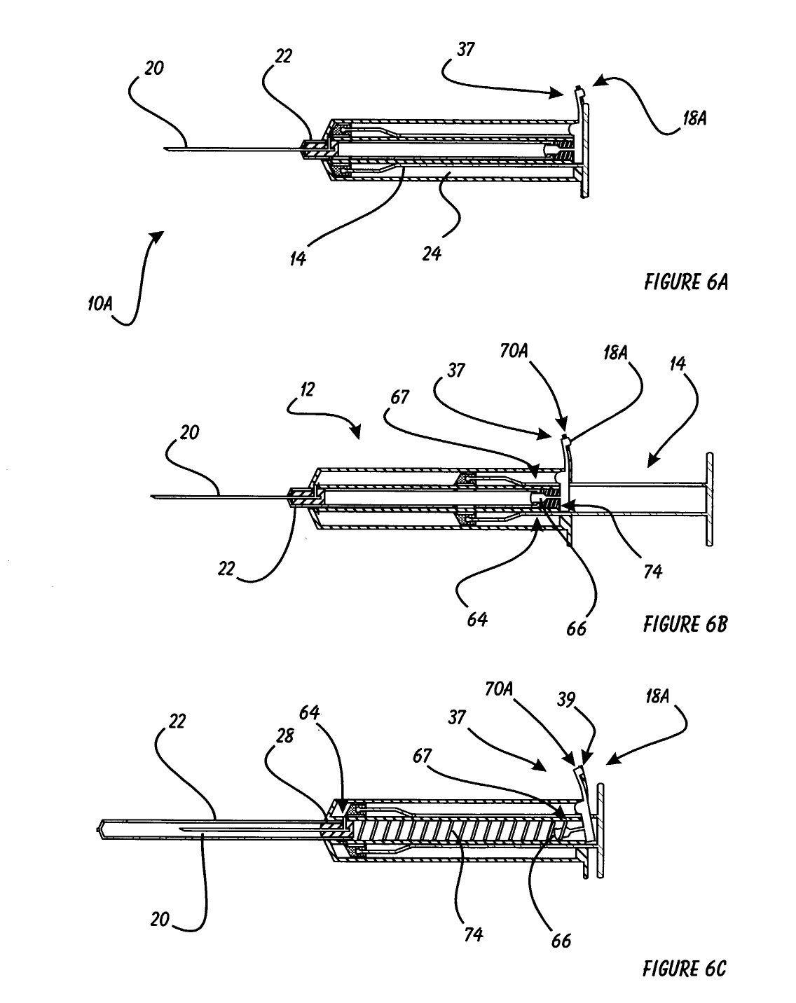

As can be seen from this cross-sectional view of the syringe 10A, the design is one of concentric circular chambers. At the outermost layer is the outer wall 32, adjacent to which is the annular reservoir 24, bounded at its inner side by an inner wall 34. Inside of the inner wall 34 is the guard chamber (see FIG. 2), within which is the guard 22. The guard 22 has a needle port slot 56 formed along much of its length to permit the radial port 30 to pass through between the annular reservoir 24 and the center bore 29, while still permitting the guard 22 to slide out of the guard chamber (see FIG. 2). The needle base 28 (and needle) is located at the very center of the syringe 10A. Having reviewed the individual components, we will now turn to FIGS. 6A-6C to discuss the operation of the present invention.

FIGS. 6A-6C are cutaway side views of the operation of the safety syringe 10A of FIG. 5. FIG. 6A depicts a cutaway side view of the syringe 10A while the plunger 14 is completely pushed in to the end of the annular reservoir 24. Also depicted here are the release guard 37 and the alternate guard release member 18A. In this embodiment, the guard 37 and release member 18A are curved or angled in the direction of the needle end of the syringe 10A; this curvature provides ergonomic comfort as well as increased safety. The curved guard 37 and release member 18A shape increase the safety of the device by separating the button on the release member 18A from the end (i.e. the end to be pressed upon) of the plunger 14, thereby inhibiting inadvertent triggering of the guard 22. While the release member 18A is separate from the plunger 14 end, it is still very conveniently located in the proximity of the plunger 14 end to make it an easy one-handed operation to trigger the guard 22 when the plunger 14 is fully or partially depressed (i.e. by sliding one's thumb from the end of the plunger 14 onto guard release member 18A).

FIG. 6B depicts the syringe 10A with the plunger 14 nearly completely retracted. Also labeled in this figure is the interaction between the ridge 64 on the open end of the guard 22 and the ridge 67 located on the guard-engaging portion 66. This engagement is what prevents the biasing member 74, which is in the compressed position here, from pushing the guard 22 to extend out to cover the end of the needle.

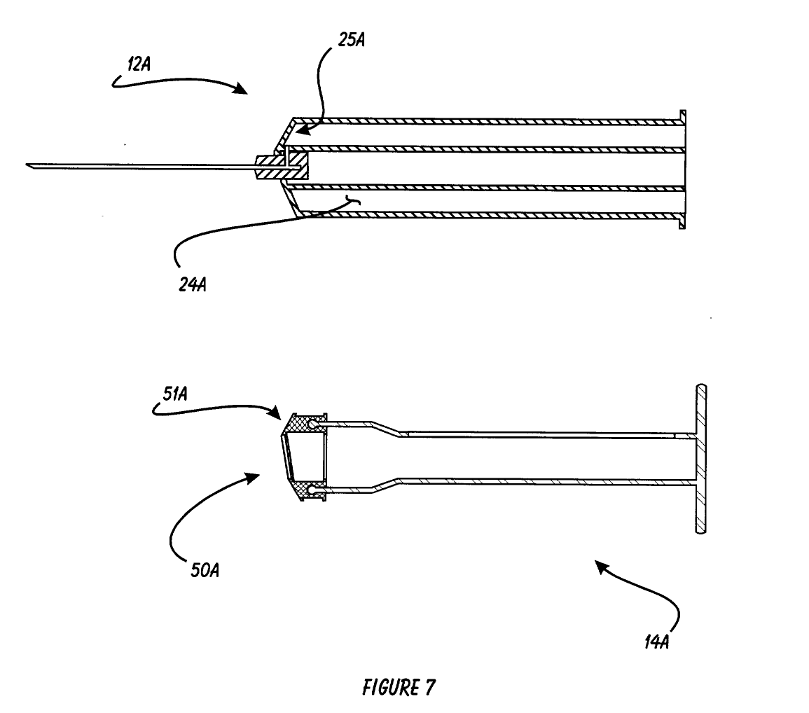

In FIG. 6C, the guard release member 18A has been depressed until the button 70A has been pushed down through the aperture 39. As the button 70A is pushed downward (as depicted here), the ridge 67 on the guard-engaging portion 66 will be released from the ridge 64 on the guard 22. Once released, the guard 22 will be forced down the length of the needle by the biasing force of the biasing member 74. Again, the ridge 64 of the guard 22 will then engage a ridge or latch located within the needle base 28; this engagement will effectively lock the guard 22 in the extended position to permanently prevent any needlesticks. Now turning to FIG. 7, we can examine an alternate design for the present invention.

FIG. 7 depicts cutaway side views of an embodiment of the asymmetrical tip versions of the body and plunger of the present invention. The modified plunger 12A here includes a modified annular reservoir 24A; this reservoir 24A is defined by an asymmetrical closed end 25A. As can be seen, the closed end 25A has a slightly longer portion of its circumference; this small volume is provided to capture the last small air bubbles that might reside in the reservoir 24A. After the bubbles are captured, it is a simple matter to expel the bubbles from the reservoir by depressing the plunger 14A slightly.

The modified plunger 14A is a standard plunger having a modified sealing ring 50A attached thereto. This sealing ring 50A has a protruding portion 51A for cooperating with the longer portion 25A of the reservoir 24A. Again, this provides increased insurance against air bubbles that may be entrained in the reservoir 24A.

Those skilled in the art will appreciate that various adaptations and modifications of the just-described preferred embodiment can be configured without departing from the scope and spirit of the invention. Therefore, it is to be understood that, within the scope of the appended claims, the invention may be practiced other than as specifically described herein.

What is claimed is:

1. A syringe, comprising:
a body defined by a guard chamber located substantially at the axial center of said body, said guard chamber surrounded by an annular reservoir, said body further defined by an open end and a needle end;
a plunger assembly, said plunger assembly comprising a plunger stem defining a longitudinal stem void, said plunger assembly terminating in a sealing ring, said sealing ring configured to be accepted by said annular reservoir.

2. The syringe of claim 1, further comprising:
a needle base located at said needle end, said needle base further comprising a radial port and a central bore.

3. The syringe of claim 2, further comprising a guard, said guard defined by a needle port slot, said needle port slot configured to accept said radial port therethrough.

4. The syringe of claim 3, further comprising a guard release member attached to said body adjacent to said open end.

5. The syringe of claim 4, further comprising a biasing member, said biasing member configured to bias said guard towards said needle end.

6. A syringe, comprising:
a body, said body defining a needle end and an open end, said body comprising:
a central guard chamber defined by an inner wall at its outer periphery and a needle base at said needle end; and
an annular reservoir surrounding said guard chamber said annular reservoir defined between said inner wall and an outer wall; and
a guard defining a guard wall, said guard wall cooperatively designed to reside in said central guard chamber.

7. The syringe of claim 6, wherein said body is further defined by a release guard further defined by an aperture formed therethrough.

8. The syringe of claim 7, further comprising a guard release member attached to said body substantially adjacent to said open end, said guard release member further comprising a button, said aperture cooperating with said button to accept said button therein.

9. A syringe, comprising:
a substantially cylindrical guard defining an outer diameter;
a substantially cylindrical body defining a longitudinal axis, a needle end and an open end, said body comprising:
an elongated guard chamber centered on said longitudinal axis and defining an outer surface, said outer surface having an outer diameter greater than said guard outer diameter, said guard insertible in said guard chamber; and
an elongated annular reservoir centered on said longitudinal axis, further having an outer diameter that is greater than said guard chamber outer diameter.

10. The syringe of claim 9, wherein:
said guard is defined by a needle end and an open end and further comprising a ridge formed adjacent to said open end; and
said syringe further comprises a guard release member, said guard release member configured to be insertible in said open end and to engage said ridge.

11. The syringe of claim 10, further comprising a needle base centered on said longitudinal axis, further having an outer diameter less than said guard outer diameter, said needle base located adjacent to said needle end.

12. The syringe of claim 11, wherein:
said needle base is further defined by a radial port in fluid communication with said annular reservoir; and
said guard is further defined by a longitudinal slot for accepting said radial port therein.

13. The syringe of claim 12, wherein said guard release member further comprises a lever having a distal end and a proximate end and a guard engaging portion extending from said proximate end.

14. The syringe of claim 13, wherein said guard release member further comprises a button protruding from said distal end.

15. The syringe of claim 14, wherein:
said annular reservoir further defines an outer wall said outer wall defining an inner surface;
said syringe further comprising a plunger assembly, said plunger assembly comprising:
a plunger stem defining first and second ends; and
a sealing ring at said second end, said sealing ring defining an inner ring surface configured to create an interference fit with said guard chamber outer surface, said sealing ring further defining an outer ring surface configured to create an interference fit with said inner surface of said outer wall of said annular reservoir.

16. The syringe of claim 15, further defined by a longitudinal slot for accepting said guard release member therein.
